(19) United States
(12) Patent Application Publication
(10) Pub. No.: US 2014/0243651 A1
(43) Pub. Date: Aug. 28, 2014

(54) HEALTH DIAGNOSIS SYSTEM USING IMAGE INFORMATION

(71) Applicants: Min Jun KIM, Seoul (KR); Se Young Kim, Chungcheongbuk-do (KR)

(72) Inventors: Min Jun KIM, Seoul (KR); Se Young Kim, Chungcheongbuk-do (KR)

(21) Appl. No.: 14/192,058

(22) Filed: Feb. 27, 2014

(30) Foreign Application Priority Data
Feb. 27, 2013 (KR) 10-2013-0021012

(51) Int. Cl. A61B 5/00

(52) U.S. Cl. CPC A61B 5/0033
USPC 600/407

(57) ABSTRACT

A health diagnosis system comprises information input unit for receiving basic information and image information for user's body part; recognition unit for recognizing the body part as object by auto search based on image information received by input unit; analysis unit drawing diagnosis results by analyzing image information recognized by the said recognition unit according to checkup elements for each body part, comparing and analyzing the analyzed information and standard information recorded in pathological information database unit; pathological information database unit for storing standard information which can diagnose names and severities of diseases by checkup elements for each body part for the analysis unit to draw the diagnosis results by comparing and analyzing the analyzed information and the standard information; and diagnosis results database unit for recording the image information received by the information input unit for user's body parts and diagnosis results drawn by the said analysis unit.

### BACKGROUND OF THE INVENTION

1. Field of the Invention

The present invention relates to health diagnosis system using image information, in particular to technology which can check and diagnose user's health condition and pathological state by drawing diagnosis results by comparing and analyzing with image information for user's body part recognized by computing device with camera with standard information for determining severity and name of disease which can be diagnosed by checkup elements.

2. Description of the Related Art

For many reasons, it is not easy for ordinary people to visit medical institutions. In an average case, people are tended not to visit medical institutions until they feel uncomfortable or pain from some problems. But if they visit medical institutions after leaving health problem untreated for some period of time, their medical expenses will be increased, remission rate will be decreased, and time for cure will take longer compared to early diagnosis/early treatment. It is very difficult for an individual to purchase a medical equipment for personal use, and it is also very difficult for an individual to use an equipment which requires expert knowledge.

Recently, remote pathological diagnosis methods have been studied in order to overcome these problems.

Regarding remote pathological diagnosis method, there are applications disclosed including Korean patent application (Korean patent publication No. 10-2002-0016289, hereinafter, ‘prior art’).

The method according to the prior art comprises a step of transmitting personal information of a patient for pathological checkup to pathological diagnosis center on the internet from client's terminal of each medical institution where the pathological checkup cannot be practiced; a step of issuing a diagnosis schedule to a client asking for checkup after listing personal information of patents transmitted from the said client's terminal in order of transmission; a step of transmitting image information of a patient required for pathological checkup from client's terminal to pathological diagnosis center; a step of practicing pathological checkup by a medical specialist with a patient's image information data deduced and image displayed by an image display equipment, etc.; a step of transmitting diagnosis results attached with the opinion of a medical specialist to a client's terminal.

However, prior art is to transmit checkup result including opinion of a medical specialist according to pathological checkup to user, and there was a problem that an extra operator or a specialist having expert knowledge for diagnosis has to monitor and pathological diagnosis is not performed quickly. Besides, currently there is no technology that recognizes body parts automatically, analyzes according to checkup elements for body part, and draws diagnosis results automatically.

### SUMMARY OF THE INVENTION

Accordingly, the present invention was devised in view of the problems above, the purpose of the present invention is to check and diagnose user's health condition with diagnosis results drawn by comparing and analyzing with image information for user's body part recognized by computing device with camera with standard information for determining severity and name of disease which can be diagnosed by checkup elements.

The present invention to achieve this technological solution relates to health diagnosis system using image information, and comprises information input unit for receiving basic information and image information for user's body part; recognition unit for recognizing the body part as object by auto search based on image information received by the information input unit; analysis unit drawing diagnosis results by analyzing image information recognized by the said recognition unit according to checkup elements for each body part, comparing and analyzing the analyzed information and standard information recorded in pathological information database unit; pathological information database unit for storing standard information which can diagnose names and severities of diseases by checkup elements for each body part in order to for the analysis unit to draw the diagnosis results by comparing and analyzing the analyzed information and the standard information; and diagnosis results database unit for recording the image information received by the information input unit for user's body parts and diagnosis results drawn by the analysis unit.

According to the present invention as described above, diagnosis results can be drawn by comparing and analyzing with image information for user's body part recognized by computing device with camera with standard information for determining names and severities of diseases which can be diagnosed by checkup elements, and it can be easily available to many people because other equipment or sensors are not needed. And also according to the present invention, recognized image information is analyzed by checkup elements for body part, based on color, shape, movement, size, secretions of body part etc. thus it has the effect that pathological diagnosis can be easily carried without monitoring of other operator or specialist, and accuracy of pathological diagnosis can be increased.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a general drawing illustrating conceptually health diagnosis system using image information according to the present invention.

FIG. 2 is an exemplary drawing showing object search/recognition algorithm in the computer vision field according to the present invention.

FIG. 3 is an exemplary drawing showing checkup elements of eye according to the present invention.

FIG. 4 is an exemplary drawing showing checkup elements of nose according to the present invention.

FIG. 5 is an exemplary drawing showing checkup elements of mouth according to the present invention.

FIG. 6 is an exemplary drawing showing checkup elements of neck according to the present invention.

FIG. 7 is an exemplary drawing showing checkup elements of chest and breast according to the present invention.

FIG. 8 is an exemplary drawing showing checkup elements of abdomen according to the present invention.

FIGS. 9 and 10 is an exemplary drawing showing checkup elements of neck according to the present invention.

FIG. 11 is an exemplary drawing showing checkup elements of buttock and anus according to the present invention.

FIG. 12 is an exemplary drawing showing checkup elements of urogenital organ according to the present invention.

FIG. 13 is an exemplary drawing showing checkup elements of upper limbs according to the present invention.

### DESCRIPTION OF SPECIFIC EMBODIMENTS

Hereinafter, a detailed description will be given of the present invention.

A better understanding of the present invention may be obtained via the following examples which are set forth to illustrate, but are not to be construed to limit the present invention, which will be apparent to persons having ordinary knowledge in the art.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

The particular characteristic and benefit of the present invention will be more clarified with the following detailed explanation based on the drawings.

Prior to this, in case a publicly known function and its specific description related to the present invention unnecessarily blur the gist of the present invention, the specific description can be omitted.

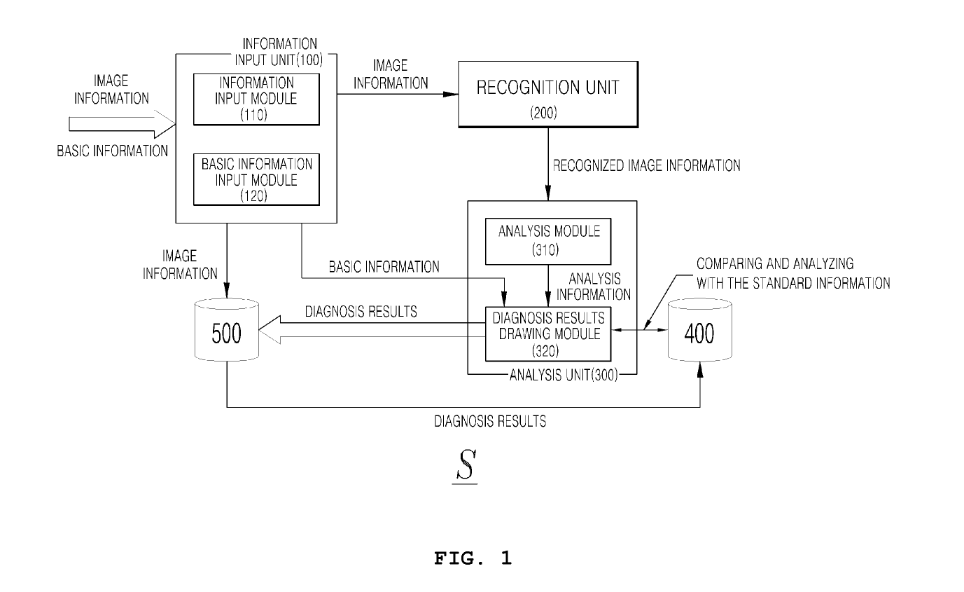

Hereinafter, the present invention is described in details in referring to the enclosed drawings. Health diagnosis system using image information according to the present invention is described is described in referring to FIGS. 1 to 13.

FIG. 1 is a general drawing illustrating conceptually health diagnosis system using image information S according to the present invention, comprises information input unit 100, recognition unit 200, analysis unit 300, pathological information database unit 400 and diagnosis results database unit 500 as shown.

Information input unit 100 performs function to receive image information for user's body part and basic information, thus includes image information input module 110 and basic information input module 120 as shown in FIG. 1.

Particularly, image information input module 110 receives image information for a user's body part. At this time, image information input module 110 can receive image information of user's whole body additionally to pre-calculate the size of body part, which can be used in determining the size of the wounded part to be analyzed by analysis unit.

The image information for body part including still image and video is communicated with outside based on pre-stored information and real-time input information, which can be input by camera mounted on computing device accessible to communication.

Basic information input module 120 receives basic information of user.

Herein, basic information of user includes body weight, age, gender, occupation, diet, living location (tropical region, temperate region, cold region, ocean, mountain, etc.), race (mongoloid, caucasian, negroid, germanic tribes, the Celts, the Han, etc.) life rhythm (wakeup time, bed time, meal time), menstrual cycle (woman), exercise quantity, smoking frequency, and past history.

Communication with outside is carried based on pre-stored information and real-time input information, which can be input by camera mounted on computing device accessible to communication.

The recognition unit 200 recognizes body part as object by auto search based on image information received through the said information input unit 100. The said recognition unit 200 can automatically search and recognize face (eye, nose, mouth, ear, forehead line, eyebrow, etc.), neck (sternal notch, clavicle, thyroid cartilage (so called Adam's apple), chest (nipple), abdomen (umbilicus, ASIS), back (spinal curvature), buttock (both gluteal sulcus, sacral curvature), upper limb (skin structure of elbow, styloid process of ulna, hand nail), pelvic limb (knee joint structure, medial and lateral maleola, toe nail).

The recognition unit 200 can preset the said body parts to automatically search and recognize, and in case auto search and recognition is failed or user wants to examine only particular region of body part, the corresponding body part or location can be searched and recognized by receiving user's control information.

In order to evaluate elements for determining user's pathology in the present invention, body part object is searched and recognized using Object Detection/Recognition algorithm.

This Object Detection/Recognition technology is used currently in many fields. For typical example when photos are taken using digital camera or smart phone camera, it is used to find a human face in the object or further take a photo automatically when a person starts smiling. Furthermore, technology which analyzes photo in document and recognize characters in it is used in name card recognition, scanner etc.

This technologies are the way they extracts specific information needed for Object Detection/Recognition using optical image information such as photo image information and analyzes and investigates based on the conventional training information.

Generally, in OpenCV (http://opencv.org/), a program library for image processing used often in computer vision area, basically several algorithms for Object Detection/Recognition are provided. The most representative algorithm is Haar Feature-based Cascade (Paul Viola and Michael J. Jones. Rapid Object Detection using a Boosted Cascade of Simple Features. IEEE CVPR, 2001, Rainer Lienhart and Jochen Maydt. An Extended Set of Haar-like Features for Rapid Object Detection. IEEE ICIP 2002, Vol. 1, pp. 900-903, September 2002).

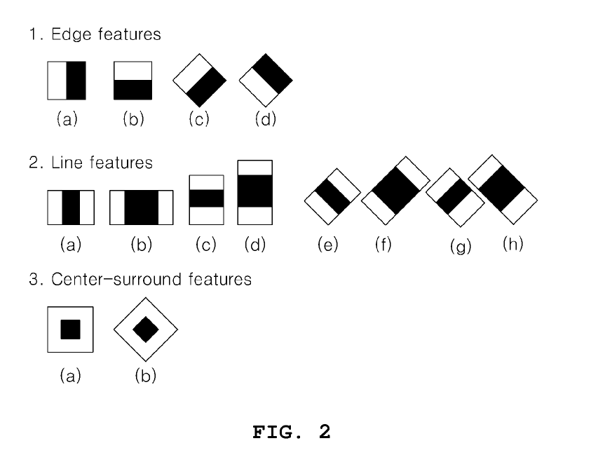

In this algorithm, array of pixel forming a photo is detected and observes as below. Therefore the region detected as object is discerned as the form made of 1—(a), 2—(b) etc. in FIG. 2, and location and enlargement/reduction information of those found region is collected additionally. Targeted object is searched by comparing the found information with conventional training data.

Analysis unit 300 performs the function drawing diagnosis results by analyzing image information recognized by the said recognition unit 200 according to checkup elements for each body part, comparing and analyzing the analyzed information and standard information recorded in pathological information database unit 400, thus includes analysis module 310, and diagnosis results drawing module 320.

Specifically, analysis module 310 analyzes image information recognized through the said recognition unit 200 by checkup elements for body part, based on color, shape, movement, size, secretions of body part, etc.

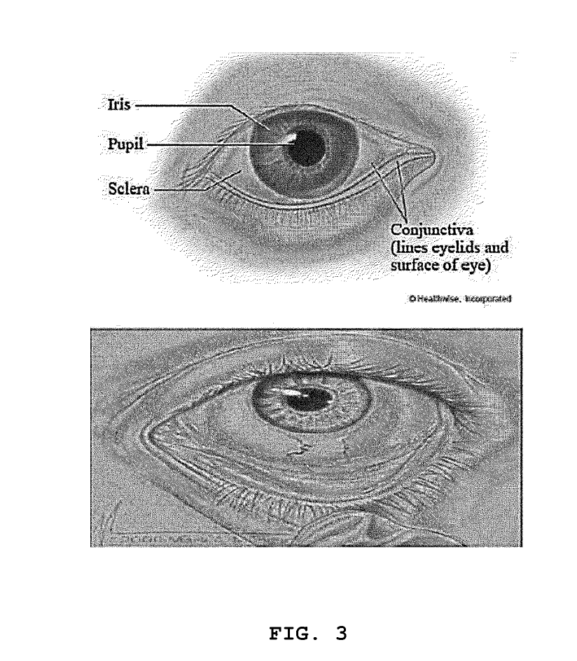

More specifically, in case of eye, analysis module 310 analyzes eyes by checkup elements such as color of iris, size of pupil, size change of pupil for light, position of eyeball, Nystagmus state, exophthalmos state, etc. (See FIG. 3).

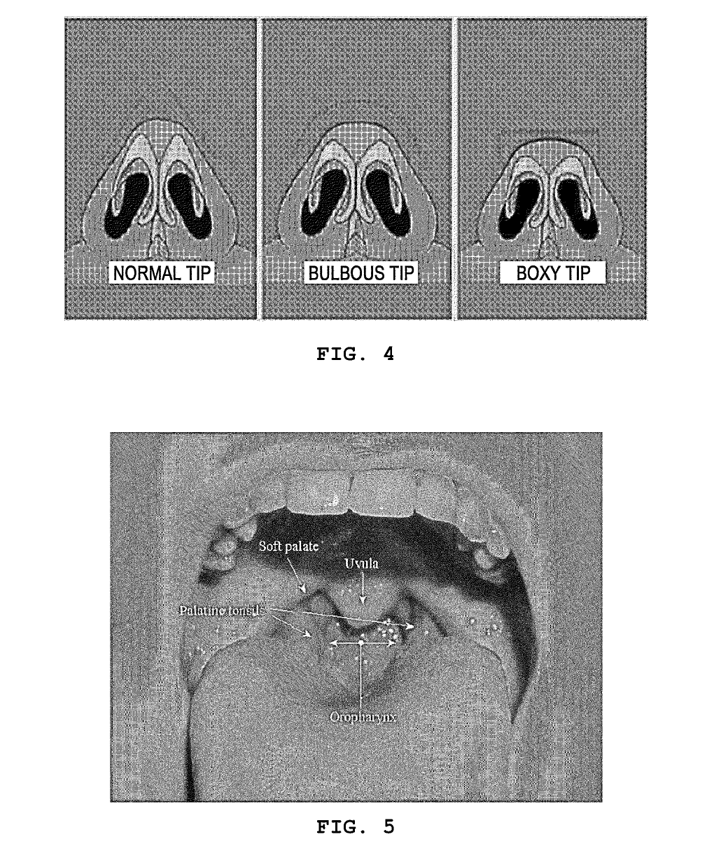

Also, in case of nose, analysis module 310 analyzes nose by checkup elements such as angle of nose and middle line, derivation of nasal bone and septum, size and shape of upper and lower lateral cartilatage, height and shape of columella, size and shape of alar, size and shape of External sidewall valve, color and viscosity of nasal mucus, etc. (See FIG. 4).

Also, in case of mouth, analysis module 310 analyzes mouth by checkup elements such as shape and array of teeth, shape and color of gum, color of tongue, color of surface of overall mouth (oral mucosa), papular lesion (macular) state, ulcerative lesion state, shape and color of lips, shape and color of skin around lips, size, shape and color of palatine tonsil, size and position of uvula, etc. (See FIG. 5).

Also, in case of face except for eyes, nose, and mouth, analysis module 310 analyzes face by checkup elements such as wrinkle position of face skin, size, distribution, color of pores, acne state, overall shape of face, shape and symmetry of auricle, shape, and protrusion state of cheek bone and forehead, and jaw, edema state of face, etc.

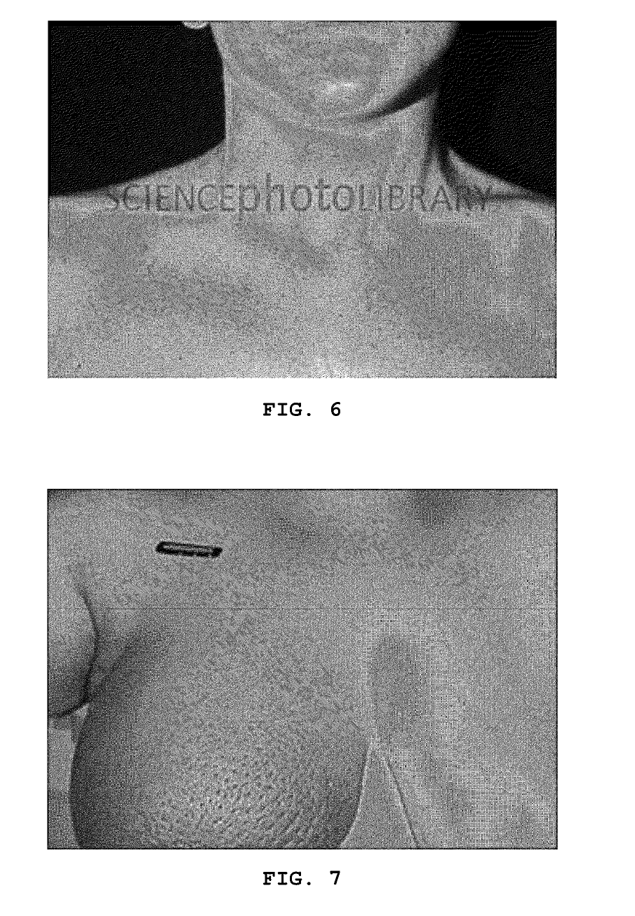

Also, in case of neck, analysis module 310 analyzes neck by checkup elements such as symmetry of both sides centered on the midline, shape and position of clavicle, position and shape of thyroid cartilage (Adam's apple), hypertrophic state of nodular mass of thyroid, tilting angle of head, etc. (See FIG. 6).

Also, in case of chest and breast, analysis module 310 analyzes chest and breast by checkup elements such as size, shape, symmetry, color of nipple and areola, color, state, and position (unilateral or bilateral)of nipple secretion, shape of male pectorialis major muscle and surrounding structure, etc. (See FIG. 7).

Also, in case of abdomen, analysis module 310 analyzes abdomen by checkup elements such as symmetry of overall abdomen, distension state, shape, size and position (position estimation is based on overall abdomen) of belly button, shape, symmetry, and size of rectus abdominis muscle of abdomen,

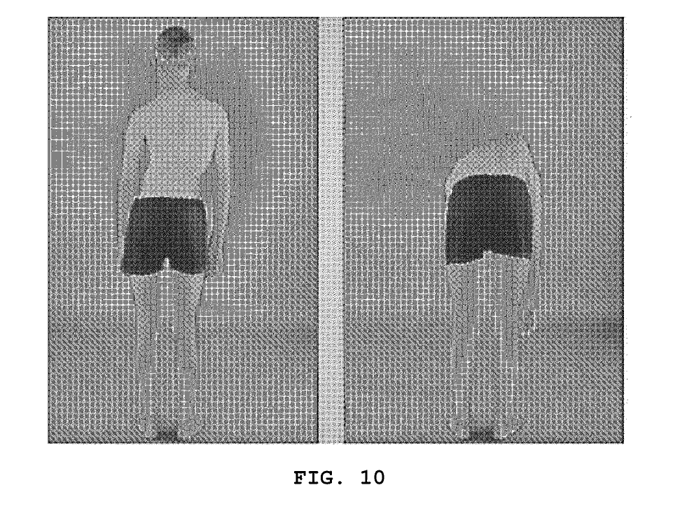

Also, in case of back, analysis module 310 analyzes back by checkup elements such as posterior view: scoliosis state, size, shape and position of back muscle, lateral view: kyphosis state, size, shape and position of abdominal protrusion, etc. (See FIGS. 9 and 10).

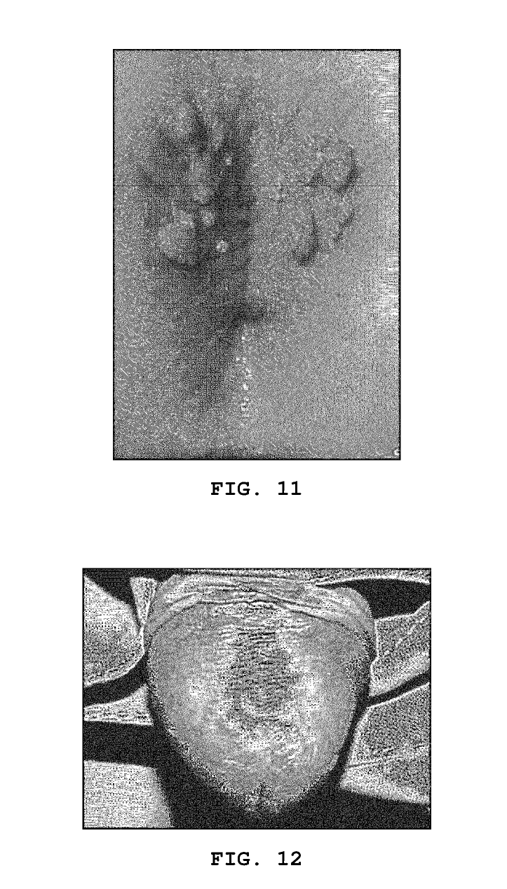

Also, in case of buttock and anus, analysis module 310 analyzes buttock and anus by checkup elements such as size, shape, and symmetry of buttock and position of gluteal sulcus, size, position(direction) and color of protrusion part of anal ring, color, area excreting secretion, protrusion and dimpling area of skin around anus, etc. (See FIG. 11).

And, in case of genitourinary organs for male, analysis module 310 analyzes genitourinary organs by checkup elements such as color, shape of glans penis, ulcer state, circumcised or uncircumcised, color and shape of urethra, secretion, etc. (See FIG. 12). For female, analysis module 310 analyzes genitourinary organs by checkup elements such as color and shape of labia majorum, ulcer state, color and shape of labia minor, color, quantity and viscosity (stickiness) of vaginal secretion, etc.

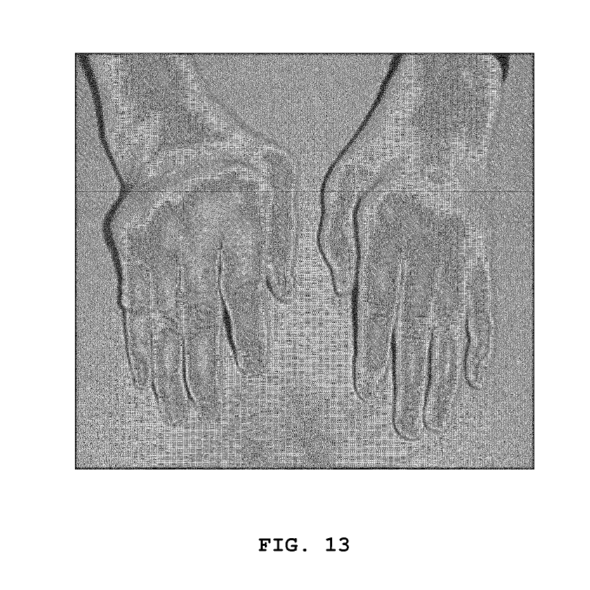

And, in case of upper extremity, analysis module 310 analyzes shoulder joint by checkup elements such as shape, size, symmetry, horizontality of both shoulders in standing posture, horizontality of both shoulders in posture with one's body leaned forward in about 90 degree, and it analyzes upper arm, elbow, forearm, and wrist by checkup elements such size, shape, and color, and it analyzes wrist joint, carpometacarpal joint, interphalangeal joint by checkup elements such as shape, and color, and it analyzes finger nail by checkup elements such as shape, size and color, etc. (See FIG. 13).

And, in case of lower extremity, analysis module 310 analyzes lower extremity by checkup elements such as shape, size, and symmetry of coxa (hip joint), size, shape, and symmetry of femoral region, shape, size, edema state, and symmetry of both sides of knee joint, shape, size, degree of curvature, and degree of protrusion of vein of calf, size, shape, and edema state of ankle joint, size, position and symmetry of both medial and lateral maleola, size, shape, degree of curvature, edema state of sole joints and toes, size, shape and color of toe nails.

And, in case of lesion area, analysis module 310 analyzes lesion area by checkup elements such as anatomical position of lesion, color of lesion, size change, and size of lesion, shape of lesion (scab state, bullous, purulent finger nail or toe nail invasive state), ulcerative state, etc.

Meanwhile, for movement, analysis module 310 analyzes using direction, distance, time of movement, etc., and for size, analysis module 310 analyzes using methods as following, and analyzes viscosity of secretion using degree of extensibility from the image; i) a method obtaining scale by using proportion per each part through image information of whole body, ii) a method using same points on multiple photos by 3D modeling, iii) a method calculating with information of a camera with which photos are taken, iv) a method photographing an object which can be scale in the same distance with the affected area simultaneously.

Diagnosis results drawing module 320 draws diagnosis results by comparing and analyzing information analyzed by the analysis module 310 and information recorded in database unit 400.

That is, diagnosis results drawing module 320 draws diagnosis results by comparing and analyzing with standard information determining names and severities of diseases which can be diagnosed by checkup elements for each body part.

For reference, in case of eye, cataract can be diagnosed if eye becomes white from cornea until pupil and iris start to change white. Also, improvement/worsening can be diagnosed by comparing degree of intrusion and past condition (See FIG. 3).

In case of nose, bridge of nose, what we call, (columella) is divided into mainly 3 kinds according to its angle and height, and there are difference in not only shape observed by the naked eye but also risk of rhinitis, sinusitis, etc. (See FIG. 4).

In case of mouth, human teeth become different in shape and number according to age. By checking the shape and number, risk of snaggle tooth, need for teeth correction, and time for treatment can be determined. Also, size change of tonsils helps to diagnose a disease for adult and infant. For example, in case only one of tonsils was enlarged, infection of tuberculosis or lymphatic lesion (in another words, a kind of leukemia) could be suspected. In case both tonsils were enlarged with ulcerative lesion observed, secondary bacterial infection from cold could be suspected to prescribe antibiotics.

And shape and position of uvula can be used in diagnosis and treatment of snoring. If it leans on one side or its size is more than 40% of throat, snoring can be caused. In practice, partial resection or position correction can be done for those who have snoring problem (In FIG. 5, Soft palate, uvula, palatine tonsils (often called tonsils), oropharynx; part between mouth and neck).

And in case of neck, thyroid in normal condition is invisible to the naked eyes, and is not touched by hand. In case of hypertrophy or nodule in thyroid, it would become visible to the eyes, and touchable by hand. And in case of overall hypertrophy, goiter can be diagnosed. Possibility of diagnosis and lesion can be varied depending on whether nodule is in one side or two sides. For an infant, it is often found a head is tilted to one side by examining the inclination of head. Many parents may overlook this to fail to recognize this as disease. In this case, toticolis can be suspected, this is caused by unbalance of SCM muscle (sternocleidomastoid muscle). If it is found in infancy, it can be corrected by physical therapy. But if it is found later than infancy, surgical operation for SCM muscle can be needed for correction. Often grandmothers find it while bathing their 3-4 year old grandchild and visit the hospital (See FIG. 6).

And, for chest and breast, women's nipple can be changed in shape and color depending on period of menstruation, pregnancy, breast-feeding, etc. In particular, color change nipple and around areola can be indirect basis to determine pregnancy (Color has changed after pregnancy or miscarriage more than once). For women's breast, breast skin can happen to change like orange peel sign. This is the aspect which can suspect breast cancer and must require consultation with a medical specialist (See FIG. 7).

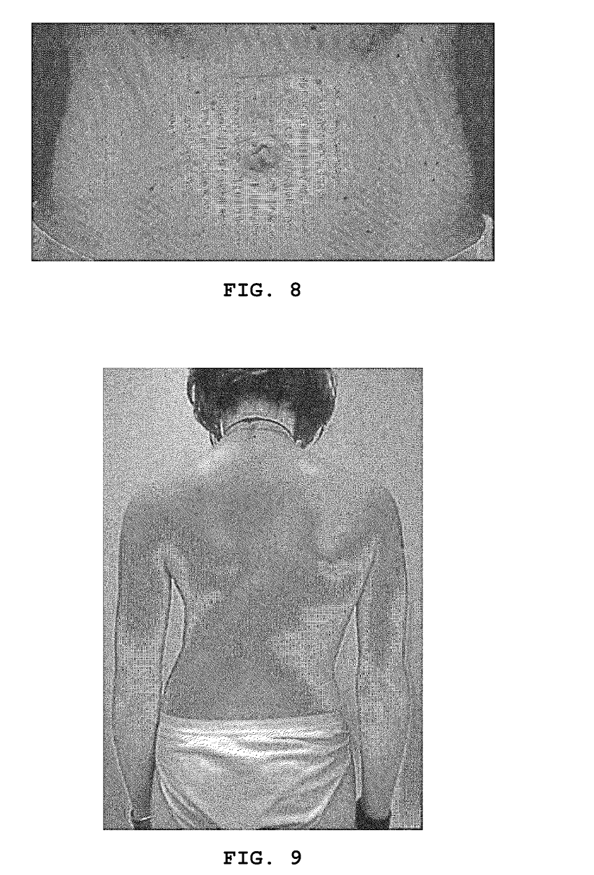

In case of abdomen, human navel varies in shape. There is umbilical hernia in some cases. Umbilical hernia is a hernia of abdominal viscera at the navel and needs a surgical treatment. If the time to find or recognize Hernia is prolonged, and size is tended to be enlarged, and the more delayed, the recurrence rate after surgical treatment rises higher (See FIG. 8).

And in case of back, in case someone is seated in comfortable posture, which looks strange or crooked, scoliosis can be suspected. And diagnosis is possible by opinion for spine with visual inspection or simple checkup (test method for scoliosis: method checking if the height of both shoulders are different, while bending forward) (See FIGS. 9 and 10).

And, for buttock and anus, in case that multiple wart-like protrusion appears around anus or genital, it is disease called condyloma acuminate or genital warts which is a kind of venereal disease. Also, protruded area around anus ring can be suspected as external hemorrhoid, internal hemorrhoid, or sometimes malignancy depending on position, size, pain state (See FIG. 11).

And for urogenital organs, skin disease occurred in mail genital can be divided into venereal disease and general dermatitis and requires different treatment. Uncleanness for long time or frequent sexual intercourse can cause balanoposthitis. Most people misperceive it as a venereal disease and visit the hospital when it grows worse. It can be cured by simple treatment, applying antibiotics and keeping clean (See FIG. 12).

And, in case of upper extremity, curve or shape change of finger is important element for Rheumatic arthritis diagnosis (See FIG. 13).

Pathological information database unit 400 stores standard information to determine names and severities of diseases which can be diagnosed by checkup element for body part in order to derive diagnosis results by comparing and analyzing with information analyzed through the analysis unit 300. The standard information is pathological information from machine learning of real diagnosis results.

Thus, pathological information database unit 400 can increase accuracy of pathological diagnosis by performing machine learning through training.

This machine learning comprises the steps of receiving diagnosis results drawn by diagnosis results drawing module 320 of the analysis unit 300 and saved in database unit 500, normalizing stored information and storing back.

At this time, with diagnosis results from diagnosing more users, more information of disease which can occur by users' characteristics can be obtained. For this, pathological database unit 400 can receive users' basic information by basic information module 120 of the information input unit 100.

Thus, pathological information unit 400 can increase reliability of diagnosis results by calculating stochastically for disease which can occur depending on gender, age, occupation and body condition using received users' basic information, and updating basic information. And there also can be a method making old data fall behind according to disease spreadable by generation, year, etc.

Diagnosis results database unit 500 stores diagnosis results drawn from the analysis unit 300 and image information for user's body part received by the information input unit 100. Also as described above diagnosis results database unit 500 can provide pathological information database 400 with diagnosis results for update of pathological information by machine learning of pathological information database unit 400.

This diagnosis result as user's pathological state information includes information about list of suspected diseases, probability, severity, tendency of improvement and deterioration of suspected diseases, etc.

The present invention can be realized as code which is computer-readable in record media which can be read by computer. The record media which can be read by computer includes all kinds of record equipment which stores computer-readable data. There are ROM, RAM, CD-ROM, magnetic tape, floppy disk, optical data storage equipment, etc. for example, as computer-readable record media, which also includes realization in carrier wave form such as transmission through internet. And also computer-readable record media is dispersed in computer system connected with network, and code which can be read by computer in dispersion method can be stored and run.

In the above, although the embodiments of the present invention have been described with reference to the accompanying drawings, a person skilled in the art should apprehend that the present invention can be embodied in other specific forms without departing from the technical spirit or essential characteristics thereof. Such appropriate changes and corrections and equivalents should be included in the scope of the present invention. Thus the embodiments described above should be construed as exemplary in every aspect and not limiting.

1. A health diagnosis system using image information, comprising:
information input unit for receiving basic information and image information for user's body part;
recognition unit for recognizing the body part as object by auto search based on image information received by the information input unit;
analysis unit for drawing diagnosis results by analyzing image information recognized by the recognition unit according to checkup elements for each body part, comparing and analyzing the analyzed information and standard information recorded in pathological information database unit; the pathological information database unit for storing standard information for determining names and severities of diseases according to checkup elements for each body part in order for the analysis unit to draw the diagnosis results by comparing and analyzing the analyzed information and the standard information; and
diagnosis results database unit for recording the image information received by the information input unit for user's body parts and diagnosis results drawn by the analysis unit.

2. The health diagnosis system of claim 1, wherein the information input unit comprises image information input module for receiving image information for a user's body part; and
basic information input module for receiving user's basic information.

3. The health diagnosis system of claim 1, wherein the user's basic information includes at least one or more among user's weight, age, gender, occupation, diet, living location, race, life rhythm, menstrual cycle, exercise quantity, smoking frequency, drinking frequency and past history.

4. The health diagnosis system of claim 1, wherein the recognition unit does auto-scan and recognizes at least one or more among face including eye, nose, mouth, ear, forehead line, eyebrow, neck (sternal notch, clavicle, thyroid cartilage (so called Adam's apple)), chest (nipple), abdomen (umbilicus, ASIS), back (spinal curvature), buttock (both gluteal sulcus, sacral curvature), upper limb (skin structure of elbow, styloid process of ulna, hand nail), pelvic limb(knee joint structure, medial and lateral maleola, toe nail), as landmark of body part.

5. The health diagnosis system of claim 1, wherein the analysis unit comprises analysis module for analyzing image information recognized by the recognition unit according to checkup elements for body part by the standard including color, shape, movement, size, secretion of body part; and
diagnosis results drawing module for drawing diagnosis results by comparing and analyzing the information analyzed by the analysis module and information recorded in pathological information database unit.

6. The health diagnosis system of claim 5, wherein the analysis module analyzes eyes by one or more of checkup elements such as color of iris, size of pupil, size change of pupil for light, shape and color of sclera, shape and color of conjunctiva, color, scar, opacity of cornea, position of eyeball, Nystagmus state, exophthalmos state.

7. The health diagnosis system of claim 5, wherein the analysis module analyzes nose by at least one or more of checkup elements such as angle of nose and middle line, derivation of nasal bone and septum, size and shape of upper and lower lateral cartilatage, height and shape of columella, size and shape of alar, size and shape of External sidewall valve, color and viscosity of nasal mucus.

8. The health diagnosis system of claim 5, wherein the analysis module analyzes mouth by at least one or more of checkup elements such as shape and array of teeth, shape and color of gum, color of tongue, color of surface of overall mouth (oral mucosa), papular lesion (macular) state, ulcerative lesion state, shape and color of lips, shape and color of skin around lips, size, shape and color of palatine tonsil, size and position of uvula.

9. The health diagnosis system of claim 5, wherein the analysis module analyzes face by at least one or more of checkup elements such as wrinkle position of face skin, size, distribution, color of pores, acne state, overall shape of face, shape and symmetry of auricle, shape, and protrusion state of cheek bone and forehead, and jaw, edema state of face.

10. The health diagnosis system of claim 5, wherein the analysis module analyzes neck by at least one or more of checkup elements such as symmetry of both sides centered on the midline, shape and position of clavicle, position and shape of thyroid cartilage (Adam's apple), hypertrophic state of nodular mass of thyroid, tilting angle of head.

11. The health diagnosis system of claim 5, wherein the analysis module analyzes chest and breast by at least one or more of checkup elements such as size, shape, symmetry, color of nipple and areola, color, status, and position (unilateral or bilateral)of nipple secretion, shape of male pectorialis major muscle and surrounding structure.

12. The health diagnosis system of claim 5, wherein the analysis module analyzes abdomen by at least one or more of checkup elements such as symmetry of overall abdomen, distension status, shape, size and position (position estimation is based on overall abdomen) of belly button, shape, symmetry, and size of rectus abdominis muscle of abdomen.

13. The health diagnosis system of claim 5, wherein the analysis module analyzes back by at least one or more of checkup elements such as posterior view : scoliosis state, size, shape and position of back muscle, lateral view: kyphosis state, size, shape and position of abdominal protrusion.

14. The health diagnosis system of claim 5, wherein the analysis module analyzes buttock and anus by at least one or more of checkup elements such as size, shape, and symmetry of buttock and position of gluteal sulcus, size, position (direction) and color of protrusion part of anal ring, color, area excreting secretion, protrusion and dimpling area of skin around anus.

15. The health diagnosis system of claim 5, wherein the analysis module analyzes genitourinary organs by at least one or more of checkup elements such as color, shape of glans penis, ulcer state, circumcised or uncircumcised, color and shape of urethra, secretion, color and shape of labia majorum, ulcer state, color and shape of labia minor, color, quantity and viscosity (stickiness) of vaginal secretion.

16. The health diagnosis system of claim 5, wherein the analysis module analyzes upper extremity by at least one or more of checkup elements such as shape, size, symmetry, horizontality of both shoulders in standing posture, horizontality of both shoulders in posture with one's body leaned forward, and it analyzes upper arm, elbow, forearm, and wrist by checkup elements such size, shape, and color, and it analyzes wrist joint, carpometacarpal joint, interphalangeal joint by checkup elements such as shape, and color, and it analyzes finger nail by checkup elements such as shape, size and color.

17. The health diagnosis system of claim 5, wherein the analysis module analyzes lower extremity by at least one or more of checkup elements such as shape, size, and symmetry of coxa (hip joint), size, shape, and symmetry of femoral region, shape, size, edema state, and symmetry of both sides of knee joint, shape, size, degree of curvature, and degree of protrusion of vein of calf, size, shape, and edema state of ankle joint, size, position and symmetry of both medial and lateral maleola, size, shape, degree of curvature, edema state of sole joints and toes, size, shape and color of toe nails.

18. The health diagnosis system of claim 5, wherein the analysis module analyzes lesion area by at least one or more of checkup elements such as anatomical position of lesion, color of lesion, size change, and size of lesion, shape of lesion (scab state, bullous, purulent finger nail or toe nail invasive state), ulcerative state.

19. The health diagnosis system of claim 5, wherein the diagnosis results drawing module draws diagnosis results by comparing and analyzing the information analyzed by the analysis module and standard information recorded in database unit.

20. The health diagnosis system of claim 1, wherein pathological information database unit stores standard information to determine names and severities of diseases according to the checkup elements for body part in order for the analysis unit to draw the diagnosis results by comparing and analyzing the analyzed information and the standard information.

21. The health diagnosis system of claim 1, wherein the standard information is pathological information by machine learning of diagnosis results.

22. The health diagnosis system of claim 1, wherein the pathological information database unit performs machine learning comprising the steps of receiving diagnosis results drawn by the analysis unit and saved in database unit, receiving diagnosis results information modulated or determined from the diagnosis results, normalizing the stored information and storing back.

23. The health diagnosis system of claim 1, the diagnosis results as user's pathological state information includes information about list of suspected diseases, probability, severity, tendency of improvement and deterioration of suspected diseases.
